(19) United States
(12) Patent Application Publication
(10) Pub. No.: US 2025/0311609 A1
(43) Pub. Date: Oct. 2, 2025

(54) ORGANIC PHOTODIODE INCLUDING ACID-FREE HOLE TRANSPORT LAYER AND METHOD OF PHOTOPLETHYSMOGRAPHY USING SAME

(71) Applicant: CHUNG-ANG UNIVERSITY INDUSTRY ACADEMIC COOPERATION FOUNDATION, Seoul (KR)

(72) Inventors: Dong Hwan WANG, Seoul (KR); Zhao YANG, Seoul (KR); Byung Gi KIM, Seoul (KR)

(21) Appl. No.: 18/829,667

(22) Filed: Sep. 10, 2024

(30) Foreign Application Priority Data
Mar. 27, 2024 (KR) 10-2024-0042119

(51) Int. Cl. H10K 85/10; A61B 5/024; C09D 5/24; C09D 7/63; C09D 125/18; C09D 165/00; H10K 30/86; H10K 85/20; H10K 85/60; H10K 101/30

(52) U.S. Cl. CPC H10K 85/1135; A61B 5/02427; C09D 5/24; C09D 7/63; C09D 125/18; C09D 165/00; H10K 30/86; H10K 85/141; H10K 85/215; H10K 85/654; H10K 2101/30

(57) ABSTRACT

An embodiment involves introducing heterocyclic 1,3-diazole (HDZ) into a PEDOT-(PSS) film, which significantly reduces the Coulomb force within the PEDOT-(PSS) and forms hydrogen bonds with PSS, thereby improving the morphology, optical properties, carrier mobility, and polymer structure of the film. The use of an optimized PEDOT-(NHDZ:PSS) film as an HTL for OPD has the effects such as better noise suppression, higher detectivity for weak light at low frequencies, wider bandwidth, and faster response speed for optical signals, and by utilizing these characteristics, the PEDOT-(NHDZ:PSS)-based OPD may successfully achieve diagnosis of cardiovascular-related diseases.

### CROSS-REFERENCE TO RELATED APPLICATIONS

This application claims the priority of Korean Patent Application No. 10⁻²⁰²⁴-0042119 filed on Mar. 27, 2024, in the Korean Intellectual Property Office, the disclosure of which is incorporated herein by reference.

### TECHNICAL FIELD

The disclosure relates to an organic photodiode and a method of photoplethysmography using the same, and more specifically, to the organic photodiode capable of improving the performance of an organic diode by improving the structural form of a film and the mobility of a carrier through an acid free hole transport layer, and reducing the corrosiveness and improving environmental-friendliness, and the method of photoplethysmography using the same. Accordingly, the cardiovascular-related disease diagnostic function can be implemented in a single pixel organodiode.

### BACKGROUND

Photodetectors have long played an important role in developing human society into a wide range of applications. They are indispensable tools that contribute to everything from space exploration to routine monitoring of human health.

Considering the rapid development of digital lifestyles, there has been a significant increase in interest in organic photodetectors, especially organic photodiodes (OPDs). OPDs are highly useful in a variety of emerging applications due to their tunable optical bandgap, low production costs, compatibility with lightweight devices, and environmental-friendliness.

In general, Photoplethysmography (PPG) devices are photoelectric sensors using light-emitting diodes (LEDs) and inorganic photodiodes, allowing for non-invasive monitoring of human health. Light of a specific wavelength emitted by the LEDs is irradiated onto the surface of human skin, where it then undergoes optical absorption, reflection, and transmission due to the effects of bones, veins, arteries, and the like.

Conventional inorganic photodiodes have been successfully applied to commercial PPG systems. However, inorganic photodetectors use semiconductors such as cadmium sulfide, lead sulfide, or gallium arsenide, which are harmful to the environment. Their production, use, and disposal processes involve toxic chemicals and hazardous waste, which can be very harmful to the human body, causing damage to the kidneys, lungs, and other organs and exhibiting strong carcinogenicity.

Therefore, there is a need for photodetectors with high performance, low toxicity, and environmentally friendly properties, particularly in PPG applications that require direct contact with human skin.

Conductive polymers are one of the common materials used to make organic photodiodes. The most typical material among conductive polymers is poly(3,4-ethylenedioxythiophene)-(polystyrenesulfonate) (PEDOT-(PSS)), which is a polymer mixture of two ionomers.

The PEDOT-(PSS) thin film has a core-shell structure that hinders inter-grain charge transfer and has high acidity. This causes damage to the skin during operation. It also leads to poor performance and limited stability of the device.

In this context, the development of more environmentally friendly and high-performance PEDOT-(PSS) is of great importance. It is important for compatibilization of OPD-based PPG sensors.

### PRIOR ART

### Patent Literature

(Patent Document 0001) KR 10⁻²⁰²⁰-0069608 A

### SUMMARY

The technical problem to be achieved by the present invention is to provide a photodetector with high performance, low toxicity, and environmentally friendly properties in PPG applications that require direct contact with human skin.

It is to provide a more environmentally friendly and high-performance PEDOT-(PSS) for compatibilization of OPD-based PPG sensors.

The technical problems to be achieved by the present invention are not limited to the above-mentioned technical problem, and still other technical problems not mentioned will be clearly understood by those skilled in the art from the description below.

In order to achieve the above technical problem, an embodiment of the present invention provides an organic photodiode including an acid-free hole transport layer, characterized by minimized acidity and improved nanostructure of the film, thereby reducing trap density and improving charge carrier mobility.

In the embodiment of the present invention, said acid-free hole transport layer may be characterized by a PEDOT-(PSS) film to which a heterocyclic 1,3-diazole is bonded.

In the embodiment of the present invention, said heterocyclic 1,3-diazole may be characterized by forming a hydrogen bond with said PEDOT-(PSS) to reduce the Coulomb force in said PEDOT-(PSS) and reorient the core-shell structure into a linear chain, thereby improving the hole transport capability

In order to achieve the above technical problem, another embodiment of the present invention provides a method for manufacturing an organic photodiode, including: preparing a mixed solution of a PEDOT-(PSS) solution and a heterocyclic 1,3-diazole solution; coating said mixed solution onto a substrate to form a mixed solution layer; coating an active layer solution onto said mixed solution layer to form an active layer; and depositing an electrode onto said active layer.

In the embodiment of the present invention, said active layer solution may be PBDTTT-EFT:PC₇₀BM.

In order to achieve the above technical problem, still another embodiment of the present invention provides a photodetection sensor including said organic photodiode.

In order to achieve the above technical problem, still another embodiment of the present invention provides a method of photoplethysmography using said photodetection sensor.

In the embodiment of the present invention, the performance of photoplethysmography may be improved due to the selectivity for pink LEDs in multiple wavelength bands

In the embodiment of the present invention, the detection may be possible based on the combined noise of noise spectral density, shot noise, and thermal noise at 1 Hz.

In the embodiment of the present invention, it may be characterized by extracting features by specific locations in a waveform through an acceleration plethysmogram and calculating them to diagnose cardiovascular disease conditions and determine blood circulation.

According to an embodiment of the present invention, the use of heterocyclic 1,3-diazole (HDZ) in the PEDOT-(PSS) film may improve the nanostructure of the film while significantly reducing its strong acidity.

The introduction of HDZ may significantly reduce the Coulomb force within PEDOT-(PSS) and form hydrogen bonds with PSS, thereby improving the morphology, optical properties, carrier mobility, and polymer structure of the film.

In addition, acid-free OPDs may ensure safety when used on human skin.

When the optimized PEDOT-(NHDZ:PSS) film is used as the HTL for OPD, it exhibits effects such as 258% lower noise suppression (Sₙ@1 Hz: 9.04×10⁻¹⁴ A Hz^{-1/2}), 243% higher detectivity for weak light at low frequencies (D*@1 Hz: 1.95×10¹² Jones), 135% wider bandwidth (f_{3dB}: 260 kHz), and 464% faster response speed for optical signals (light on: 0.96 s), compared to the conventional devices.

Utilizing these characteristics, PEDOT-(NHDZ:PSS)-based OPD may successfully achieve diagnosis of cardiovascular-related diseases.

It should be understood that the effects of the present invention are not limited to the above-described effects, but include all effects that may be inferred from the configuration of the invention described in the detailed description or the claims of the present invention.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A-1D are a schematic diagram showing structural changes caused by PEDOT-(PSS) polymer and HDZ (pH-surfactant) (FIG. 1A), a schematic diagram showing the hydrogen bonding mechanism between PEDOT-(PSS) and HDZ (FIG. 1), a graph showing the pH value of the PEDOT-(PSS) solution (FIG. 1C), and a graph showing the polymerization degree of the PEDOT-(PSS) sample (FIG. 1D);

FIGS. 2A-2D are schematic diagrams showing the structure of the OPD at various concentrations of HDZ (FIG. 2A), the energy level arrangement of the device structure (FIG. 2B), the actual setting using an organic photodetector (OPD) to measure photoplethysmographic signals (FIG. 2C), and the operating mechanism for diagnosing cardiovascular disease with the OPD (FIG. 2D);

FIGS. 3A-3F are graphs showing the bias-dependent current under dark conditions (FIG. 3A), the shot noise limit detectivity (Dₛₕ*) at −0.5 V of the OPD (FIG. 3B), the stability of the shot noise limit detectivity at −0.5 V for 14 days (FIG. 3C), and the linear dynamic range (LDR) of the OPD (FIGS. 3D-3F);

FIGS. 4A-4F are graphs showing the frequency-dependent response of the OPD (FIGS. 4A-4C), the transient photoresponse with the light on (FIG. 4D), and the results of signal-to-noise ratio (SNR) analysis of the OPD in high (1000 Hz) and low (1 Hz) frequency bands (FIGS. 4E and 4F);

FIGS. 5A-5F are a graph showing the noise spectrum of the instrument background and OPD (FIG. 5A), a graph showing the comparison of thermal and shot noise for the OPD (FIG. 5B), a graph showing the noise equivalent power (NEP) obtained at 1 Hz (FIG. 5C), and a 2D contour plot showing the specific detection rate (D*) as a function of the incident wavelength and frequency (FIGS. 5D-5F); and

FIGS. 6A-6E are a schematic view of the acid-free OPD-based PPG sensor for diagnosing cardiovascular diseases (FIG. 6A), graphs showing the comparison of the original (FIG. 6B), first derivative (FIG. 6C), and second derivative (FIG. 6D) of PPG signals measured using PEDOT-(NHDZ:PSS)-based OPD, and an enlarged curve showing FIGS. 6B-6D for extracting features in the cardiac cycle (FIG. 6E).

### DETAILED DESCRIPTION

Hereinafter, preferred embodiments of the disclosure, which may specifically achieve the aspects as described above, are described with reference to the accompanying drawings. However, the present invention may be embodied in a variety of different forms, and thus is not limited to the embodiments described herein. In addition, in the drawings, parts unrelated to the description are omitted in order to clearly explain the present invention, and similar parts are assigned similar drawing reference numerals throughout the specification.

Throughout the specification, when a part is said to be “connected (joined, contacted, bonded)” with another part, it encompasses not only cases where it is “directly connected”, but also cases where it is “indirectly connected” with another member interposed therebetween. Also, when a part is said to “include” a component, it means that other components may be further provided, rather than excluding other components, unless otherwise specified.

The terminology used herein is for the purpose of describing particular embodiments only and is not intended to be limiting of the invention. The singular forms “a,” “an,” and “the” include plural referents unless the context clearly dictates otherwise. In this specification, terms such as “include” or “have” should be understood as specifying the presence of features, numbers, steps, operations, components, parts, or combinations thereof described in the specification, not excluding in advance the possibility of the presence or addition of one or more other features or numbers, steps, operation, components, parts or combinations thereof.

Hereinafter, embodiments of the present invention will be described in detail with reference to the accompanying drawings.

The terms used herein are defined as follows.

“HDZ” means a heterocyclic 1,3-diazole.

“PEDOT-(NHDZ:PSS)” means PEDOT-(PSS) in which 0.5 wt % of HDZ is introduced.

“PEDOT-(BHDZ:PSS)” means PEDOT-(PSS) in which 1.1 wt % of HDZ is introduced.

The organic photodiode including the acid-free hole transport layer according to an embodiment of the present invention will be described.

The organic photodiode including the acid-free hole transport layer according to an embodiment of the present invention includes the acid-free hole transport layer, and may minimize acidity and improve nanostructure of the film, thereby reducing trap density and improving charge carrier mobility.

Said acid-free hole transport layer may be a PEDOT-(PSS) film to which a heterocyclic 1,3-diazole is bonded.

Pristine PEDOT is an organic semiconductor material. It has limited solubility in commonly used solvents, and to overcome this, PSS may be included. This provides advantageous solubility properties for PEDOT.

PEDOT-(PSS) is a conductive polymer known for its excellent transparency, compatibility, flexibility, and low production costs. In organic photodiodes, PEDOT-(PSS) is often used. PEDOT-(PSS) serves as a hole transport layer in a p-type semiconductor, and it functions to reduce leakage current injection and improve hole transport.

The PEDOT-(PSS) polymer have a granular shape. They have a structure in which a conductive PEDOT core is enclosed by a PSS shell. The insulating PSS shell hinders inter-grain charge transfer, which limits the performance of OPD using PEDOT-(PSS) as HTL or EBL. The high acidity of Pristine PEDOT-(PSS) also remains an important issue. This causes skin damage during surgery and also leads to poor performance and stability limitations of the device. In this context, developing more environmentally friendly and high-performance PEDOT-(PSS) is important for the compatibilization of OPD.

Accordingly, in the present invention, heterocyclic 1,3-diazole (HDZ) was added to induce structural changes in PEDOT-(PSS).

Said heterocyclic 1,3-diazole may form a hydrogen bond with said PEDOT-(PSS) to reduce the Coulomb force in said PEDOT-(PSS) and reorient the core-shell structure into a linear chain, thereby improving the hole transport capability.

Initially, a group of hydroxyl groups (—OH) present in the PSS structure liberates hydrogen ions (H⁺) in the aqueous medium (PSSH) (PSS⁻+H⁺). These H⁺ ions then react with the double bonds in the thiophene groups in PEDOT to promote hydrogen bond formation. At the same time, the Coulomb force between the positively charged carbon and the negatively charged oxygen in PEDOT-(PSS) results in the formation of ionic bond between these two atoms.

After the introduction of HDZ, the ionic bonding in the PEDOT-(PSS) material is gradually weakened, leading to a relaxation of the entangled structure. Accordingly, a π-π stacked structure is formed in the PEDOT-(PSS) thin film due to hydrogen bonding between the PSS and the HDZ. The structure is redirected to a linear type of PEDOT chain and HDZ while creating an improved conductive PEDOT domain.

As a result, when the organic compound PEDOT-(NHDZ:PSS) was synthesized by introducing HDZ, HDZ functions as a surfactant with the ability to adjust the pH value of the film. The pH value of PEDOT-(PSS) depends on the amount of pH adjusting surfactant added. Therefore, the pristine PEDOT-(PSS) up to pH 1.7 may be neutralized with a neutral solution that is harmless to the human body.

As a result, neutral PEDOT-(NHDZ:PSS) with excellent hole transport and electron injection blocking properties may be successfully manufactured.

Hereinafter, the method for manufacturing the organic photodiode according to another embodiment of the present invention will be described.

The method for manufacturing the organic photodiode according to an embodiment of the present invention may include: preparing a mixed solution of a PEDOT-(PSS) solution and a heterocyclic 1,3-diazole solution; coating said mixed solution onto a substrate to form a mixed solution layer; coating an active layer solution onto said mixed solution layer to form an active layer; and depositing an electrode onto said active layer.

The PEDOT-(PSS) solution and HDZ are mixed and stirred.

This mixed solution is coated onto a substrate. The substrate may be a pre-patterned ITO/glass substrate, which may be sonicated and washed with deionized water, acetone, and isopropyl alcohol (IPA). Hydrophilicity may be improved through UVOzone treatment. The coating may be carried out by spin coating. The solvent may then be evaporated through annealing for solvent removal.

The active layer solution may then be coated. Said active layer solution may be PBDTTT-EFT:PC₇₀BM. It constitutes a photosensitive layer, having a broad light absorption range and capable of producing a high EQE at 450-750 nm.

OPDs with different PEDOT-(PSS) films were fabricated with the structure of ITO/PEDOT-(PSS)/PBDTTT-EFT:PC₇₀BM/Al. The active layer solution may be prepared by adding PBDTTT-EFT:PC₇₀BM to a solution of chlorobenzene (CB) and diiodooctane (DIO).

Finally, after vacuum-treating the substrate coated with the active layer for 30 minutes, the electrode may be deposited.

Hereinafter, the photodetection sensor according to still another embodiment of the present invention will be described.

The photodetection sensor according to an embodiment of the present invention may include said organic photodiode.

The quantitative values, as compared to the conventional devices, will be described using the organic photodiode.

In the case of roughness, the root-mean-squared (RMS) value is at least 0.381 nm, with the optimized film achieving a minimum value of 0.381 nm, representing a 143% reduction in roughness and a more planarized film surface compared to the conventional one.

In the case of the optical bandgap, it ranges from 1.97 to 2.04 eV, and when optimized, the maximum value of 2.04 eV may represent an increase in the level of 0.07 eV compared to that of the conventional one.

For carrier mobility, it ranges from 1.95×10⁻³ to 5.02×10⁻³ cm² V⁻¹ s⁻¹, and when optimized, the maximum value of 5.02×10⁻³ cm² V⁻¹ s⁻¹ may represent an improvement of 257% compared to the conventional one.

For the degree of polymerization, it ranges from 78.79 to 91.04%, with the maximum value of 91.04% being that increased by 116% compared to that of the conventional one.

After 12 hours (overnight), improvements in performance may be made compared to conventional inorganic photodiodes or organic photodiodes, such as reduced corrosion inhibition.

In addition, the LUMO level may be −3.08 to −3.13 in the case of LUMO compared to the conventional condition, and a LUMO level (−3.08 eV) lower by 0.05 eV of PEDOT-(NHDZ:PSS) may prevent charge injection, reduce dark current density, reduce trap density, and improve carrier mobility. Due to these developments, the photodetection capacity (D*@1 Hz) may be 4.41×10¹¹ to 1.95×10¹² Jones, and under optimized conditions, the maximum value of photodetection capacity (D*@1 Hz: 1.95×10¹² Jones) may be greatly improved to a level of 243% compared to that of the conventional one.

The physicochemical and structural changes in PEDOT-(PSS) result in the optical signal bandwidth (f_{3db}) ranging from 192.54 to 260 kHz, with the optimized condition achieving a bandwidth of 260 kHz, which is 135% higher than that of the conventional one.

It is also possible to provide an optimized device with a response time that is 0.96 μs or more for light on, more specifically from 0.96 to 4.45 μs, and 0.96 μs for optimization, that is 464% faster compared to that of the conventional conditions.

The performance limit of the practical application will be described by the overall noise limit according to the frequency. Thus, the optimized OPD may suppress noise even at low frequencies.

This improved functionality directly contributes to the performance improvement of OPDs in practical applications, so that PPG detection systems fabricated using multi-wavelength LEDs that are well matched to OPD operating wavelengths may produce more accurate PPG waveform curves. High-precision PPG waveforms allow the system to successfully identify various features related to the cardiovascular circulation of humans, ultimately diagnosing cardiovascular diseases in a single pixel OPD.

More details will be described later in Experimental Examples again.

Hereinafter, the method of photoplethysmography according to still another embodiment of the present invention will be described.

The method of photoplethysmography according to an embodiment of the present invention may be one using said photodetection sensor.

PPG has been developed to help monitor human health. PPG is a non-invasive method of measuring changes in blood volume of skin microvessels based on optical properties such as absorption, scattering, and transmission of human body components in light of specific wavelengths. In clinical settings, PPG often employed high-performance inorganic photodetectors to measure changes in heart rate, blood oxygen saturation, and peripheral vascular tension for diagnosing heart diseases and the like. In order to develop PPG technology that is more safe and highly portable, research has been conducted to use thin film organic photodiodes for health monitoring.

Traditional inorganic photodiodes have been successfully employed in commercial PPG systems. Recently, emerging organic photodetectors have also been used in such systems. OPDs are highly valuable in a variety of emerging applications due to their tunable optical bandgap, low production costs, excellent compatibility with lightweight devices, and environmental-friendliness. They are expected to replace existing inorganic photodetectors in fields such as biomedical sensing and imaging, and wearable biometric monitors.

However, the performance of OPD in practical applications, particularly in PPG measurements, is difficult to evaluate and needs improvement. Most OPDs may only perform basic heart rate and blood oximetry. Therefore, the development of OPDs with higher performance for PPG measurements is important.

Accordingly the performance of photoplethysmography may be improved due to the selectivity for pink LEDs in multiple wavelength bands using the OPD of the present invention.

Description will be made in comparison with the conventional conditions.

The noise spectral density at 1 Hz may be greater than or equal to 9.04×10⁻¹⁴ A Hz^{-1/2}, and specifically 9.04×10⁻¹⁴ A Hz^{-1/2} to 2.33×10⁻¹³ A Hz^{-1/2}. The minimum value, which is the optimum value, is suppressed by 258% compared to that of the conventional one.

The shot noise may be greater than or equal to 6.25×10⁻¹⁵ A Hz^{-1/2}, and specifically 6.25×10⁻¹⁵ A Hz^{-1/2} to 7.84×10⁻¹⁵ A Hz^{-1/2}, and the minimum value being the optimum value is a value further reduced by 125% compared to that of the conventional one.

The thermal noise may be greater than or equal to 2.11×10⁻¹⁴ A Hz^{-1/2}, and specifically 2.11×10⁻¹⁴ A Hz^{-1/2} to 4.23×10⁻¹⁴ A Hz^{-1/2}, and the minimum value being the optimum value is a value further reduced by 122% compared to that of the conventional one.

Detection based on such combined noise may be possible.

The detectivity (D*@1 Hz) for weak light at low frequencies may be 4.41×10¹¹ Jones to 1.95×10¹² Jones, and the maximum value may have 243% higher detectivity than the conventional one.

Acceleration plethysmograms may be used to extract features by specific locations in a waveform and calculate them, to diagnose cardiovascular disease conditions and determine blood circulation.

Hereinafter, manufacturing examples and experimental examples of the present invention will be described in detail.

### Manufacturing Examples

### Synthesis of PEDOT-(PSS) Solution

Different weight ratios of HDZ were added and stirred for 20 minutes to prepare PEDOT-(PSS) solutions with different pH values. In the case of PEDOT-(PSS), PEDOT-(NHDZ:PSS) and PEDOT-(BHDZ:PSS), the weight ratios of HDZ used were 0, 0.5 and 1.1 wt. %, respectively, and no additional dopants or additives were used.

### Fabrication of Photodetection Sensor (Photodetector)

The pre-patterned ITO/glass substrate (15×15 mm²) was sonicated with deionized water, acetone, and isopropyl alcohol (IPA) for 20 minutes respectively before cleaning the solvent with nitrogen gas. The substrate was then treated with UVOzone for 15 minutes to improve hydrophilicity. OPDs with different PEDOT-(PSS) films were fabricated with a structure of ITO/PEDOT-(PSS)/PBDTTT-EFT:PC₇₀BM/Al. After filtering through a 0.45 μm PVDF (polyvinylidene fluoride) filter, PEDOT-(PSS) for each condition was spin coated at 5000 rpm on an ITO/glass substrate. The solvent was then evaporated by annealing at 140° C. for 10 minutes for removal. The active layer solution was prepared by adding PBDTTT-EFT:PC₇₀BM (25 mg/mL) to a solution of CB (1 mL) and DIO (3 μL). The active layer was fabricated in a glove box (active area: 0.15 cm²) filled with high purity argon on a PEDOT-(PSS) substrate. Finally, the substrate coated with the active layer was vacuum-treated for 30 minutes, and then a 100 nm aluminum electrode was deposited onto the device using a thermal evaporation method at 2.0×10⁻⁶ Torr with a shade mask.

### Experimental Examples

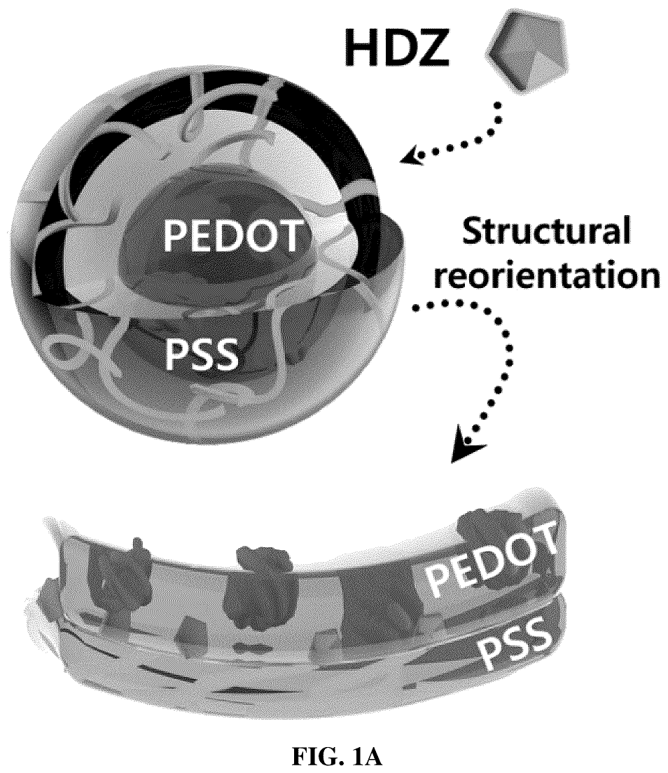

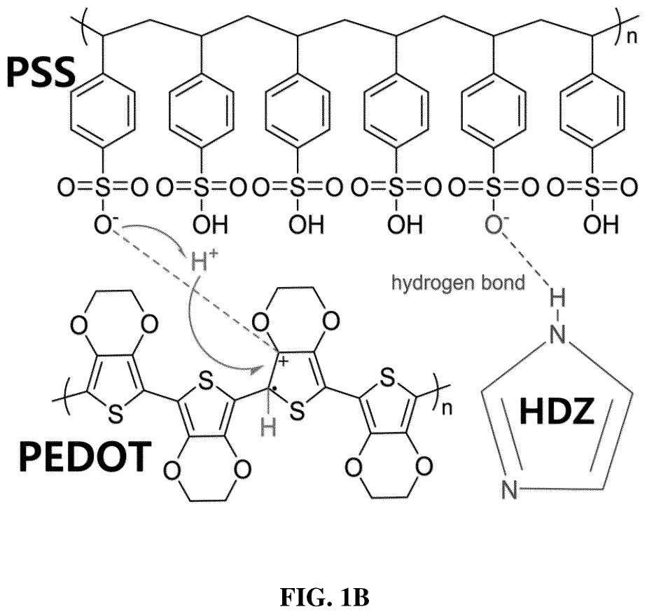

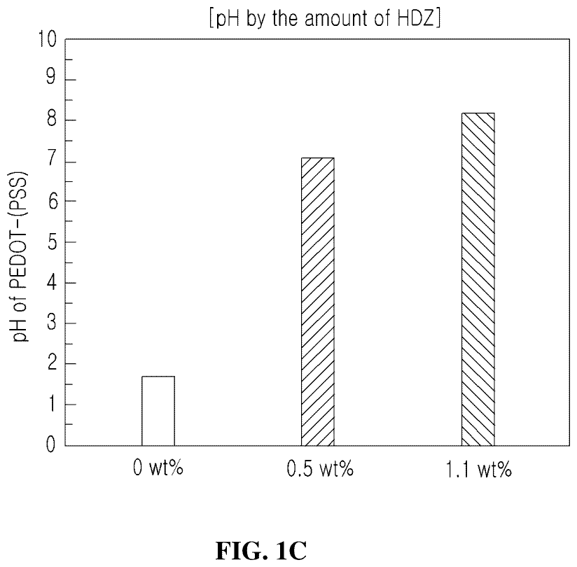

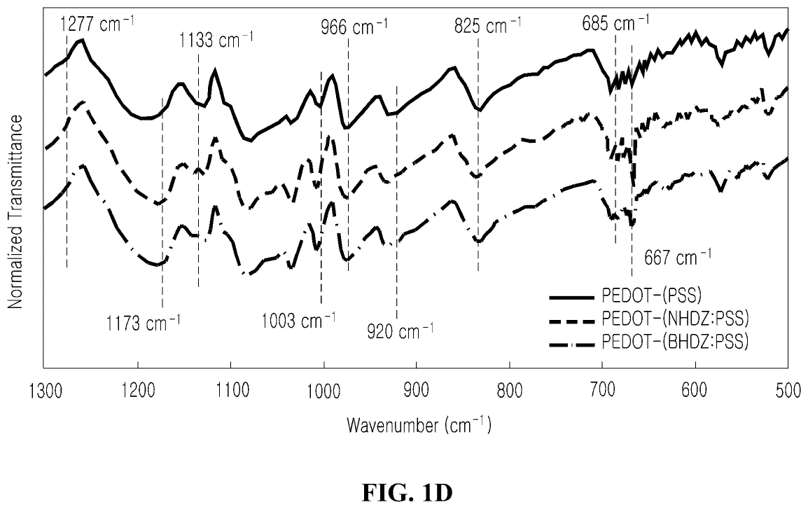

FIGS. 1A-1D are a schematic diagram showing structural changes caused by PEDOT-(PSS) and HDZ (pH-surfactant) (FIG. 1A), a schematic diagram showing the hydrogen bonding mechanism between PEDOT-(PSS) and HDZ (FIG. 1i), a graph showing the pH value of the PEDOT-(PSS) solution (FIG. 1C), and a graph showing the polymerization degree of the PEDOT-(PSS) sample (FIG. 1D).

Referring to FIG. 1A, it may be confirmed that the structure is characterized by a conductive PEDOT core enclosed by a hydrophilic PSS shell, wherein subsequent addition of HDZ results in a structural change of PEDOT-(PSS), and that structure is redirected to a linear type of PEDOT chain and HDZ while creating an improved conductive PEDOT domain.

Referring to FIG. 1B, hydroxyl (—OH) groups initially present in the PSS structure liberate hydrogen ions (H⁺) in the aqueous medium (PSSH↔PSS⁻+H⁺), and these H⁺ ions react with the double bonds in the thiophene groups in PEDOT to promote hydrogen bond formation. At the same time, it may be confirmed that the Coulomb force between the positively charged carbon and the negatively charged oxygen in PEDOT-(PSS) results in the formation of ionic bond between these two atoms (PEDOT+PSS-H⁺↔PEDOT+PSS⁻+H⁺+e⁻). It may be confirmed that after the introduction of HDZ, the ionic bonding in the PEDOT-(PSS) material is gradually weakened, so that the entangled structure is gradually relaxed, and a π-π stacked structure is formed in the PEDOT-(PSS) thin film due to hydrogen bonding between PSS and HDZ, resulting in an improvement in charge mobility.

Referring to FIG. 1C, it may be confirmed that HDZ as a surfactant, also has pH-adjusting properties, and the pH of PEDOT-(PSS) depends on the amount (0, 0.5, and 1.1 wt. %) of the added pH surfactant. The pH values of 1.7, 7.1, and 8.2 correspond to PEDOT-(PSS), PEDOT-(NHDZ:PSS), and PEDOT-(BHDZ:PSS), respectively.

FIG. 1D shows a qualitative analysis of the structural change of PEDOT-(PSS) with the addition of HDZ using FT-IR spectroscopy.

Table 1 shows the parameters for polymerization calculation in the FT-IR measurements.

TABLE 1

| | PEDOT- | PEDOT- | PEDOT- |
|---|---|---|---|
| PEDOT of PEDOT-(PSS) | (PSS) | (NHDZ:PSS) | (BHDZ:PSS) |
| R (Ratio of integrated areas | 0.72 | 0.35 | 0.69 |
| @825 cm−1/@ 625 cm−1) | | | |
| Degree of polymerization = | 0.79 | 0.91 | 0.79 |
| 2(R0/R + 2) | | | |
| Degree of polymerization (%) | 78.79 | 91.04 | 79.44 |

Referring to this, typical bands for each sample were observed around wavenumbers of 1277, 1173, 1133, 1003, 966, 920, 825, and 685 cm⁻¹. The HDZ band corresponds to 1277 cm⁻¹. The S═O and O—S—O symmetric elongation modes in the PSS correspond to 1190 and 1003 cm⁻¹. C—S—C coupling oscillations are observed around wavenumbers of 966, 920, 825, and 685 cm⁻¹. In addition, the degree of PEDOT polymerization was analyzed by integrating the peak intensity ratio based on the peaks at 825 and 685 cm⁻¹.

For the degree of polymerization, it ranges from 78.79 to 91.04%, with the maximum value of 91.04% being that increased by 116% compared to that of the conventional one.

The increased degree of polymerization is associated with crosslinking of the polymer chains due to the interaction between the additive and PEDOT-(PSS). As a result, an optimized value (≈91%) was observed in PEDOT-(NHDZ:PSS). However, excessive increase in HDZ results in irregular shapes due to unintended aggregation. This suggests that the degree of polymerization in PEDOT-(NHDZ:PSS) increased, demonstrating the improvement in electrical properties and device stability by HDZ under PEDOT-(NHDZ:PSS) conditions, which in turn leads to a stabilized form and an improved PEDOT domain.

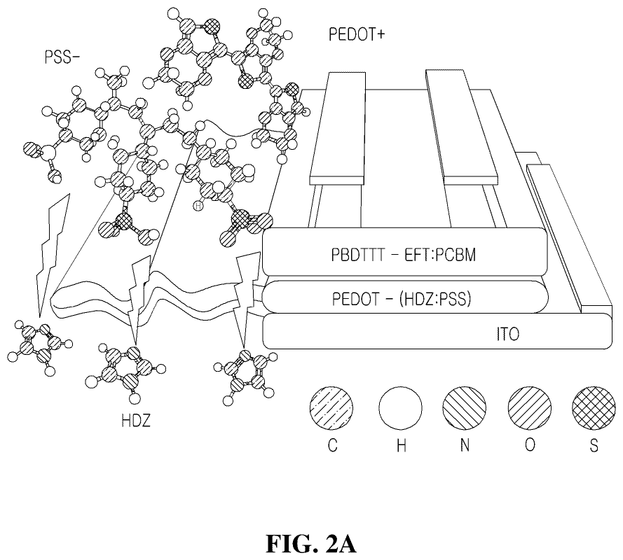

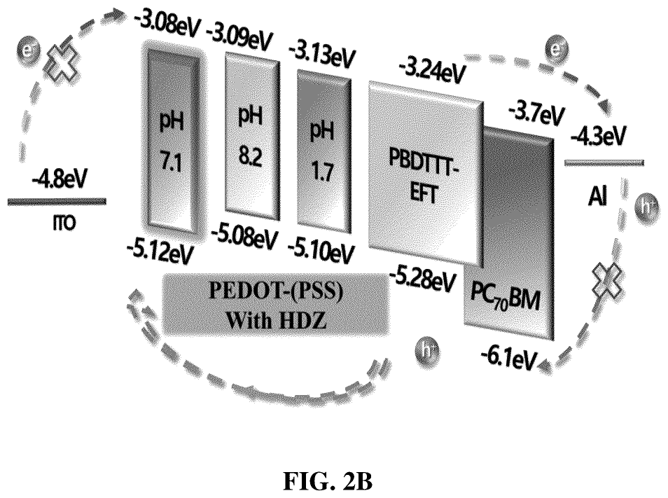

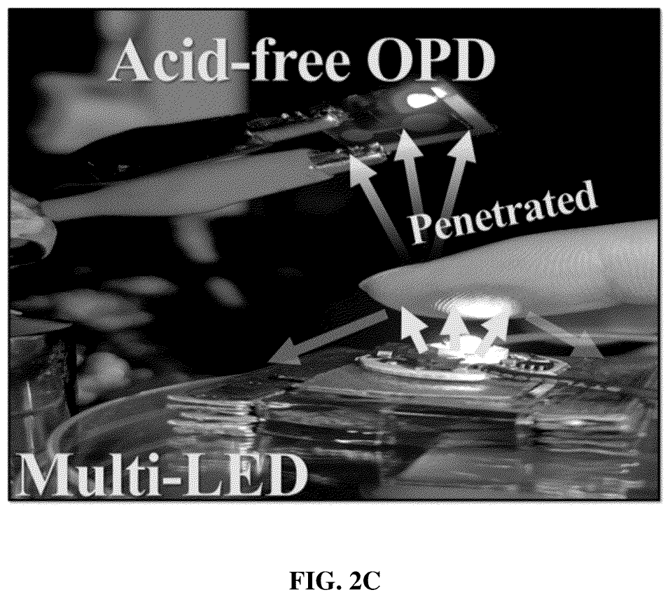

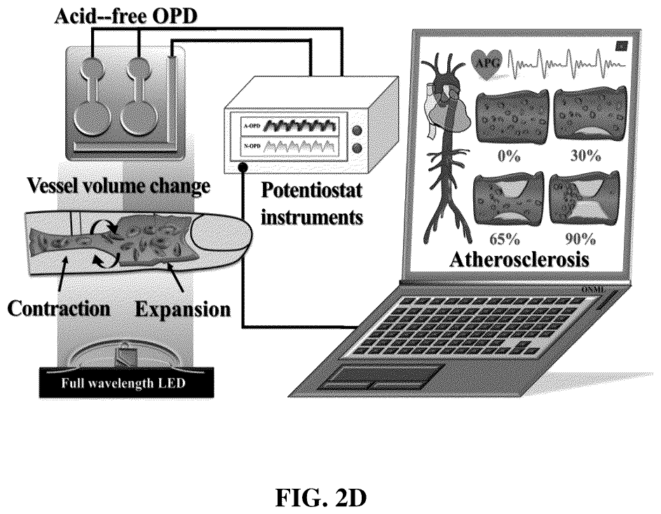

FIGS. 2A-2D are schematic diagrams showing the structure of the OPD at various concentrations of HDZ (FIG. 2A), the energy level arrangement of the device structure (FIG. 2B), the actual setting using an organic photodetector (OPD) to measure photoplethysmographic signals (FIG. 2C), and the operating mechanism for diagnosing cardiovascular disease with the OPD (FIG. 2D).

Referring to FIG. 2A, the device structure, including PEDOT-(PSS) ionically bonded to HDZ, may be confirmed.

Table 2 shows the energy levels of the PEDOT-(PSS) samples.

TABLE 2

| Each | W_{f} | Optical E_{g} | LUMO |
|---|---|---|---|
| PEDOT-(PSS) | [eV] | [eV] | [eV] |
| PEDOT-(PSS) | −5.10 | −1.97 | −3.13 |
| PEDOT-(NHDZ:PSS) | −5.12 | −2.04 | −3.08 |
| PEDOT-(BHDZ:PSS) | −5.08 | −1.99 | −3.09 |

Referring to Table 2 and FIG. 2B, energy level data was provided by cyclic voltammetry analysis performed to compare the work function of HTL samples at each pH.

The work function was −5.08 to −5.12 eV, and the optical bandgap was 1.97 to 2.04 eV. In particular, the deepest work function (−5.12 eV) and the lowest unoccupied molecular orbital (LUMO) energy (−3.08 eV) were found under PEDOT-(NHDZ:PSS) conditions. In the case of LUMO, when optimized, the maximum value of 2.04 eV represent an increase in the level of 0.07 eV compared to the conventional one. This suggests that the modification of PEDOT-(PSS) via HDZ addition induces electron injection barrier properties and dark current leakage suppression.

Referring to FIG. 2C, the light emitted by the multi-wavelength LED penetrates or is absorbed by human tissue, and the blood volume of the fingertip changes during each cardiac cycle. This results in a change in the intensity of light penetrating the fingertip. The OPDs are used to detect and extract the pulse signals, which are superimposed on the low frequency signals. This is done to evaluate heart rate. Additionally, using OPDs with a PEDOT-(NHDZ:PSS) layer, a relatively accurate original PPG waveform may be obtained. This makes it possible to analyze the original PPG curve to diagnose cardiovascular-related diseases.

Referring to FIG. 2D, it shows the working principle of PPG, and it also includes a schematic diagram of atherosclerosis characterized by the accumulation of substances such as fat and cholesterol in the arterial walls. Accurate diagnosis of atherosclerosis may be achieved by analyzing the original PPG curve and then examining the shape of the accelerated thymus (APG) waveform.

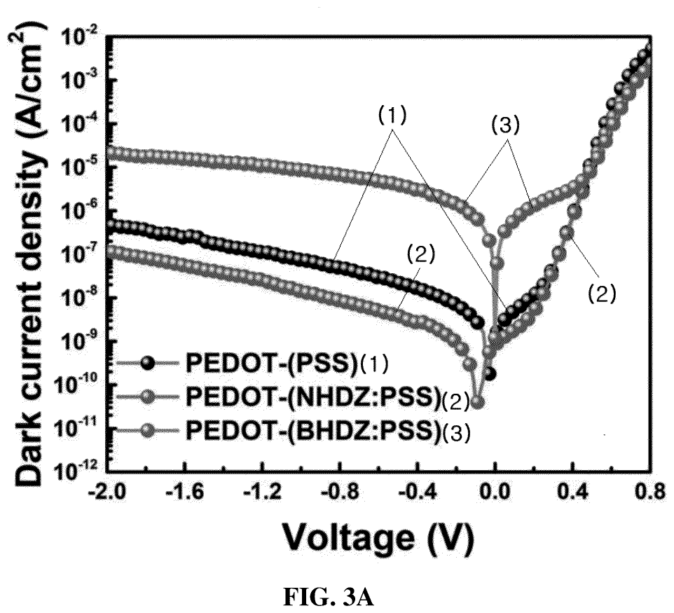

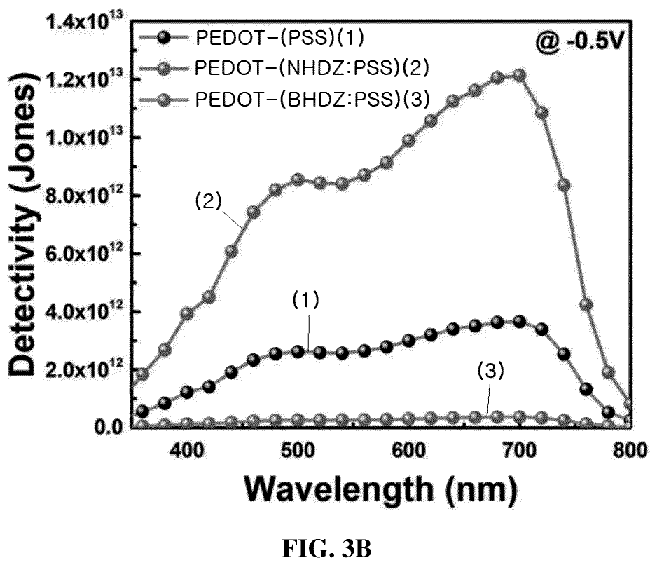

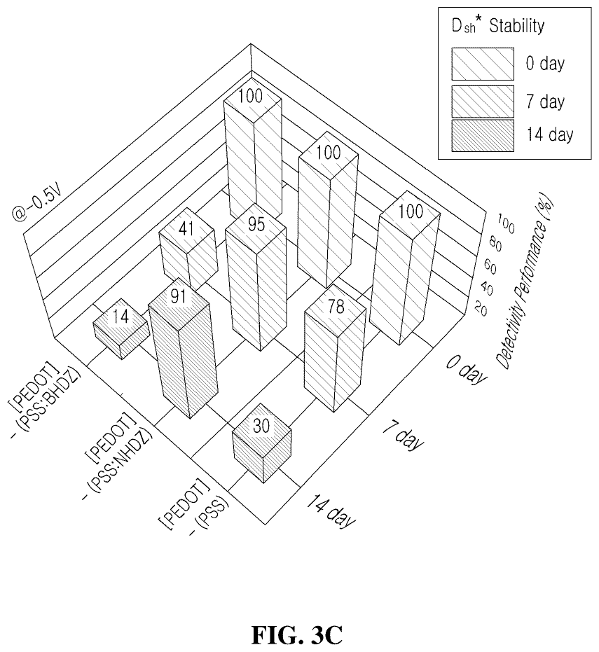

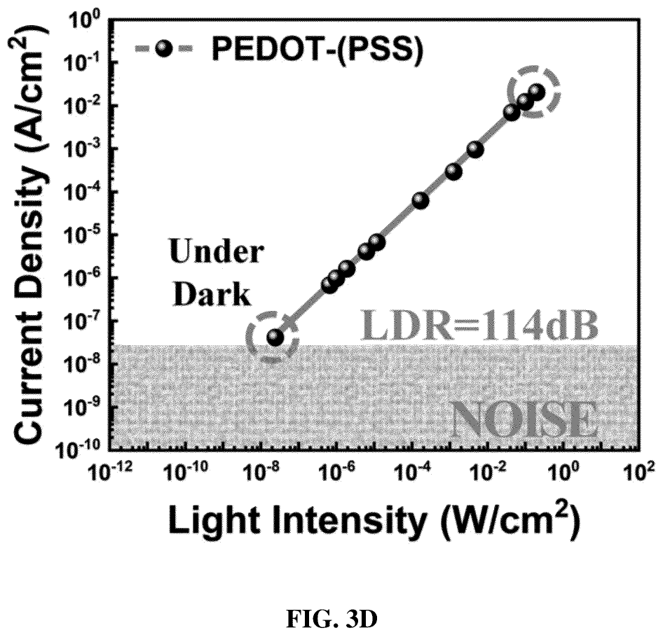

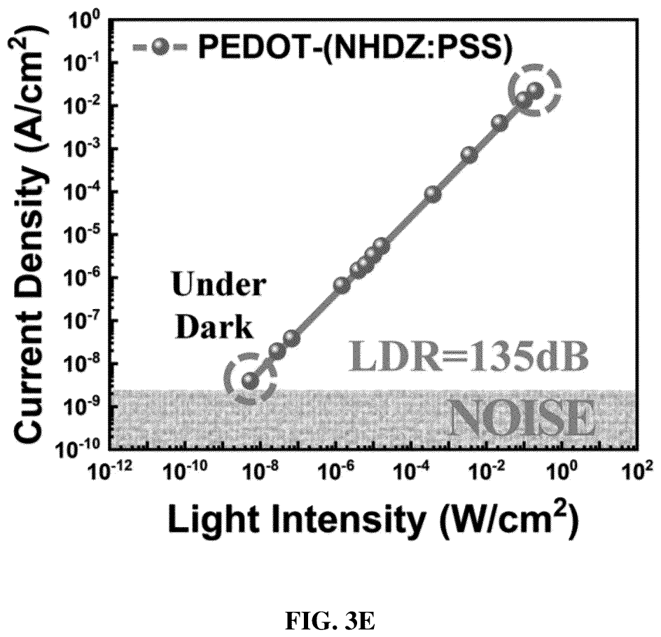

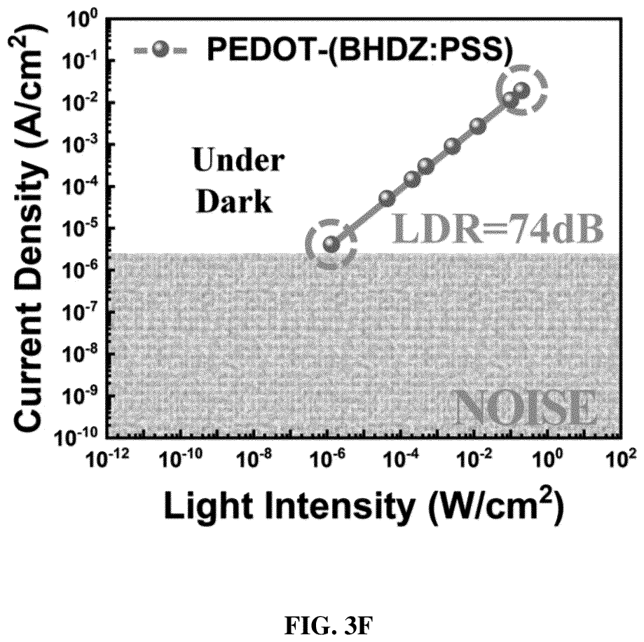

FIGS. 3A-3F are graphs showing the bias-dependent current under dark conditions (FIG. 3A), the shot noise limit detectivity (Dₛₕ*) at −0.5 V of the OPD (FIG. 3B), the stability of the shot noise limit detectivity at −0.5 V for 14 days (FIG. 3C), and the linear dynamic range (LDR) of the OPD (FIGS. 3D-3F).

Table 3 is the performance parameters of each photodetector according to HDZ content.

TABLE 3

| | J_{D} | R @ 700 nm | Dₛₕ* @ 700 nm |
|---|---|---|---|
| | under −0.5 V | under −0.5 V | under −0.5 V |
| Each OPD | (A/cm²) | (A/W) | (Jones) |
| PEDOT-(PSS) | 4.11 × 10⁻⁸ | 0.420 | 3.66 × 10¹² |
| PEDOT-(NHDZ:PSS) | 3.91 × 10⁻⁹ | 0.429 | 1.21 × 10¹³ |
| PEDOT-(BHDZ:PSS) | 4.09 × 10⁻⁶ | 0.420 | 3.67 × 10¹¹ |

Referring to FIG. 3A and Table 3, it may be confirmed that the change was clearly indicated when comparing the dark current of the OPD according to the conditions. The most significant improvement in shunt resistance with voltage change was observed under PEDOT-(NHDZ:PSS) condition. This is because the electron charge injection blocking property was effectively improved according to the change in the energy band of PEDOT-(NHDZ:PSS) compared to the PEDOT-(PSS) sample. Therefore, a dark current (3.91×10⁻⁹ A cm⁻²) at −0.5 V, which is 1051% lower, was observed compared to 4.11×10⁻⁸ A cm⁻² under the conventional conditions.

Referring to FIG. 3B, overall, the best photodetection performance across all wavelength was observed in the PEDOT-(NHDZ:PSS) sample.

Referring to FIG. 3C, when comparing the durability of the OPD for 14 days, the smallest change was observed under the PEDOT-(NHDZ:PSS) condition. This change was noted in dark current and shot noise-limited detectability.

Referring to FIGS. 3D, 3E and 3F, they show the photocurrent dependence on the input light intensity (radiance) at 630 nm for each PEDOT-(PSS) device operating at −0.5 V bias. Compared to the conventional devices, the PEDOT-(NHDZ:PSS) OPD exhibits an LDR of 135 dB increased by 118%, which is comparable to many commercial inorganic photodetectors (Si or InGaAs). Although all OPDs similarly have an upper linear limit of LDR for photocurrent, the suppressed noise charge injection in PEDOT-(NHDZ:PSS)-based OPDs allows a lower linear limit of the LDR.

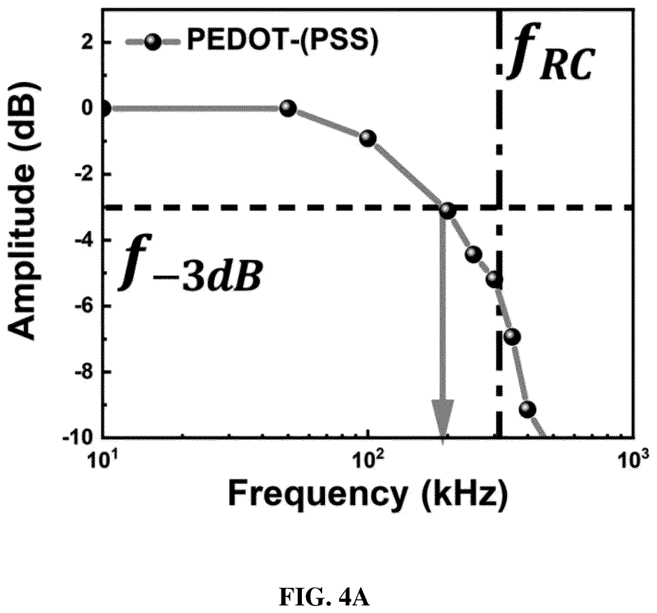

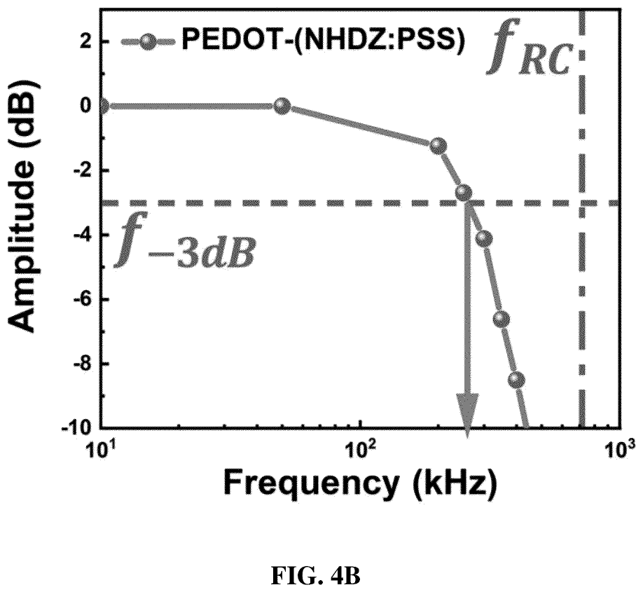

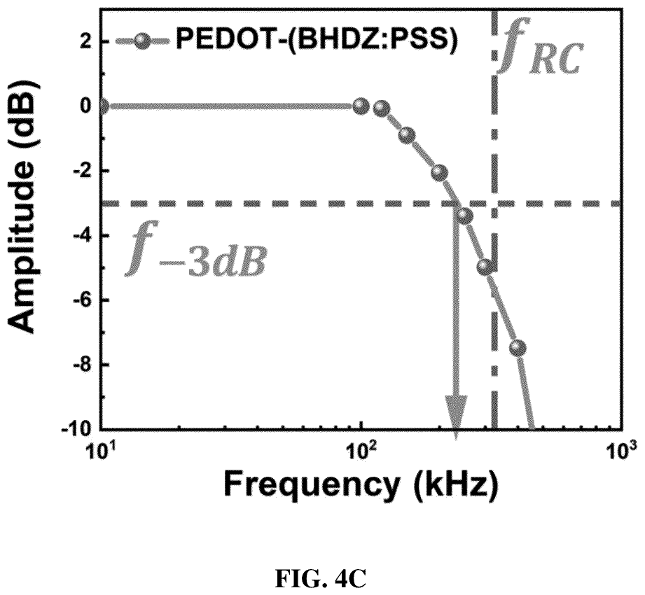

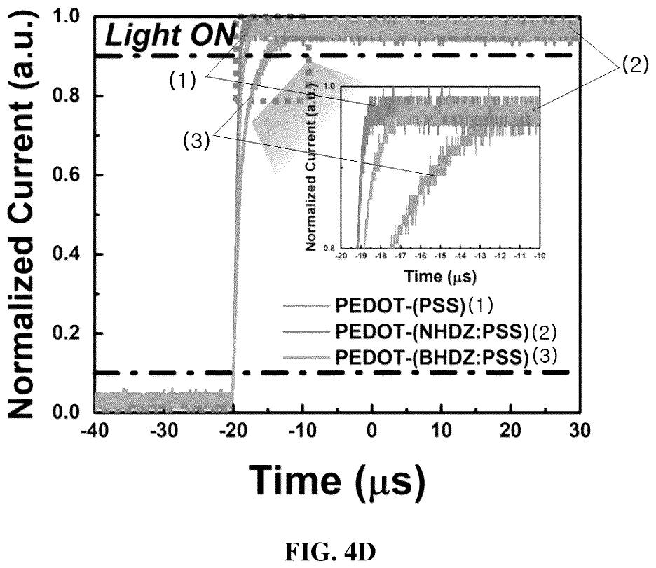

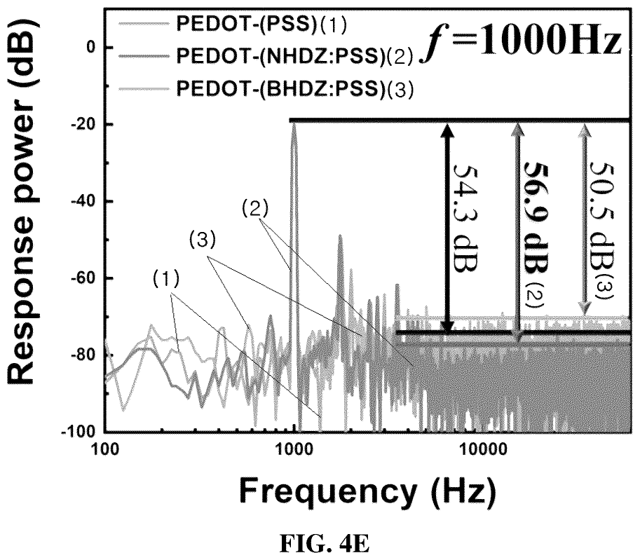

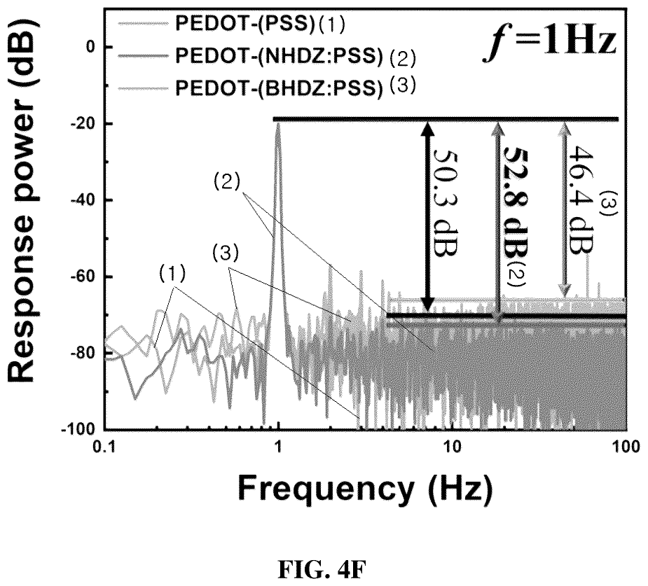

FIGS. 4A-4F are graphs showing the frequency-dependent response of the OPD (FIGS. 4A-4C), the transient photoresponse with the light on (FIG. 4D), and the results of signal-to-noise ratio (SNR) analysis of the OPD in high (1000 Hz) and low (1 Hz) frequency bands (FIGS. 4E and 4F).

Table 4 shows the transient photoresponse, bandwidth, and OPD equivalent circuit-related parameters of OPDs based on HDZ content.

TABLE 4

| | τ_{R} [symbol] | Light on | Light off | f_{RC} | f [symbol] _{B} | R [symbol] ₕ | R [symbol] | C [symbol] |
|---|---|---|---|---|---|---|---|---|
| Each PEDOT-(PSS) | (μs) | (μs) | (μs) | (kHz) | (kHz) | (Ω) | (Ω) | (F) |
| PEDOT-(PSS) | 1.05 | 4.45 | 32.48 | 312 | 193 | 2.49 × 10 [symbol] | 8.89 | 8.66 × 10 [symbol] |
| PEDOT-(NHDZ:PSS) | 0.43 | 0.96 | 29.60 | 714 | 260 | 3.68 × 10 [symbol] | 12.41 | 3.57 × 10 [symbol] |
| PEDOT-(BHDZ:PSS) | 0.98 | 1.68 | 32.16 | 325 | 234 | 9.21 × 10 [symbol] | 10.21 | 8.10 × 10 [symbol] |

[symbol]

indicates data missing or illegible when filed

Referring to FIGS. 4A, 4B and 4C, the f_{3dB} of the PEDOT-(NHDZ:PSS)-based OPD is 260.26 kHz, which is higher than that of the PEDOT-(PSS)-based OPD (192.54 kHz) and the PEDOT-(BHDZ:PSS)-based OPD (233.83 kHz), and has an optical signal bandwidth that is 135% higher than that of the conventional one.

Referring to FIG. 4D, it shows the transient photoreaction of each OPD under 452 nm LED illumination at 0 bias voltage. The OPD using the pure PEDOT-(PSS) film exhibits a rise time of 4.45 μs (output signal changed from 10% to 90% of maximum output value) and a fall time of 32.48 μs (output signal changed from 90% to 10% of maximum output value), which is consistent with the performance of the PCBM-type materials. The photoreaction of devices using PEDOT-(NHDZ:PSS)/PEDOT-(BHDZ:PSS) films exhibits 464%/265% faster rise times of 0.96 μs/1.68 μs and 110%/101% faster fall times of 29.60 μs/32.16 μs than PEDOT-(PSS)-based devices, respectively.

Referring to FIGS. 4E and 4F, the signal is converted into a fast Fourier transform (FFT) signal by an oscilloscope, and first, the LED signal is set to 1000 Hz to avoid excessive interference due to 1/f noise in the low frequency band. PEDOT-(NHDZ:PSS)-based OPD showed 105%/113% better noise suppression than PEDOT-(BHDZ:PSS)-based OPD (50.5 dB) and PEDOT-(PSS)-based OPD (54.3 dB), achieving a high SNR of 56.9 dB. The signals required for the PPG device to monitor heart beats are considered to be distributed in the range of 0.5-10 Hz. Signals in this range often have interference by 1/f noise at low frequencies. Therefore, as shown in FIG. 4F, the LED signal is set to 1 Hz, so that the OPD signal and white noise may be compared more directly. The low-frequency SNRs corresponding to the OPD are 50.3 dB (PEDOT-(PSS)), 52.8 dB (PEDOT-(NHDZ:PSS)), and 46.4 dB (PEDOT-(BHDZ:PSS)), respectively. All SNRs have similar signal peak intensities. This is because they have similar photocurrents (reactivity). The high signal quality of the PEDOT-(NHDZ:PSS)-based OPD generally results in stronger noise suppression due to lower charge trap density and higher LUMO. The device has an excellent signal detection function for both high-frequency and low-frequency signals. These characteristics make it well applicable to wearable health monitoring and other applications that require detection of weak optical signals.

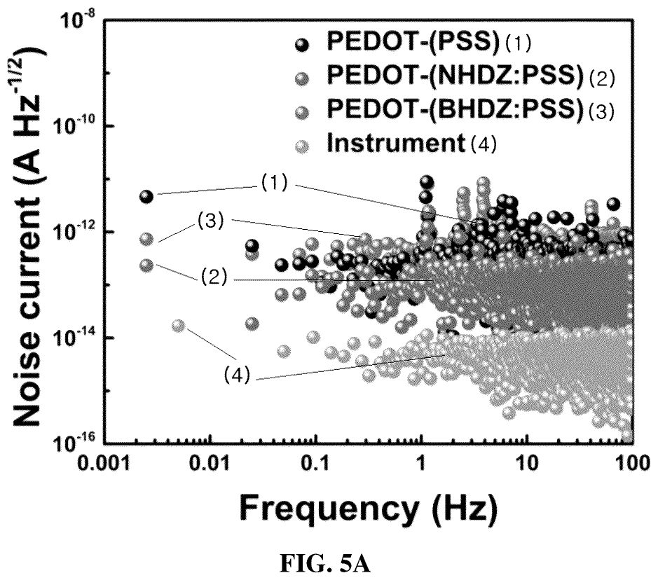

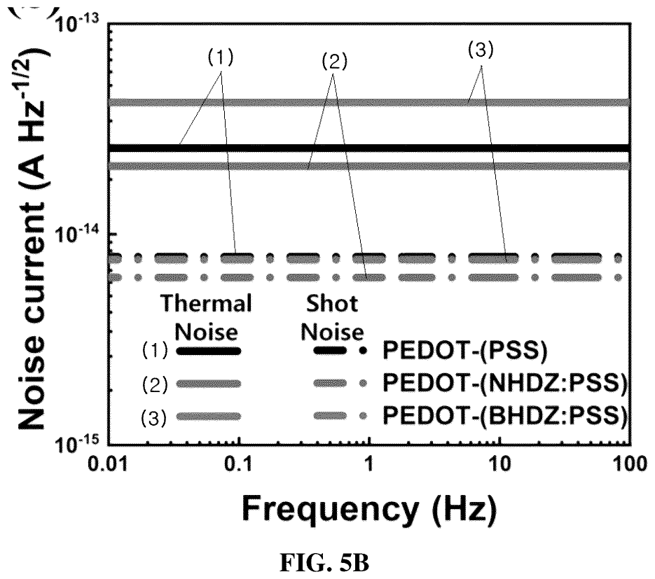

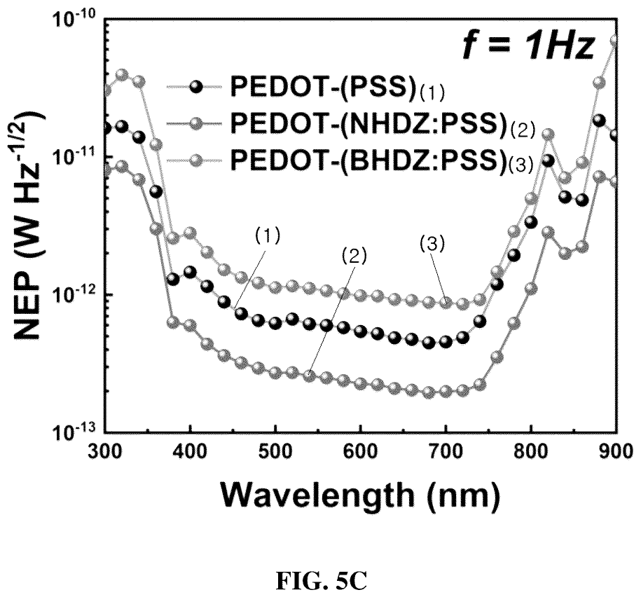

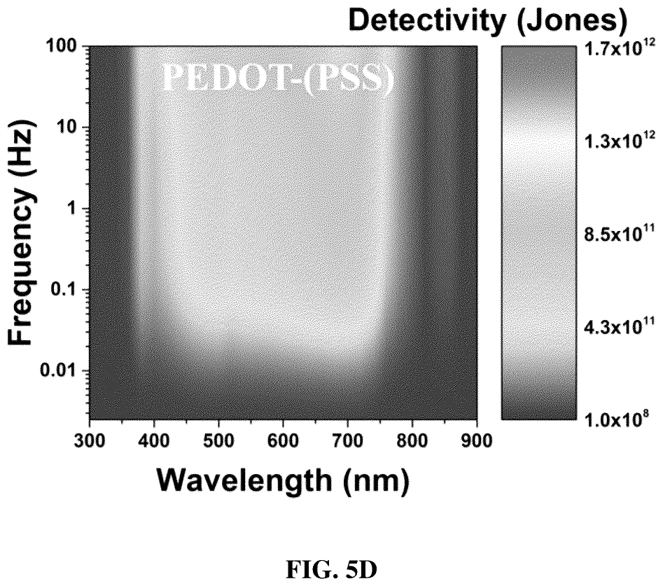

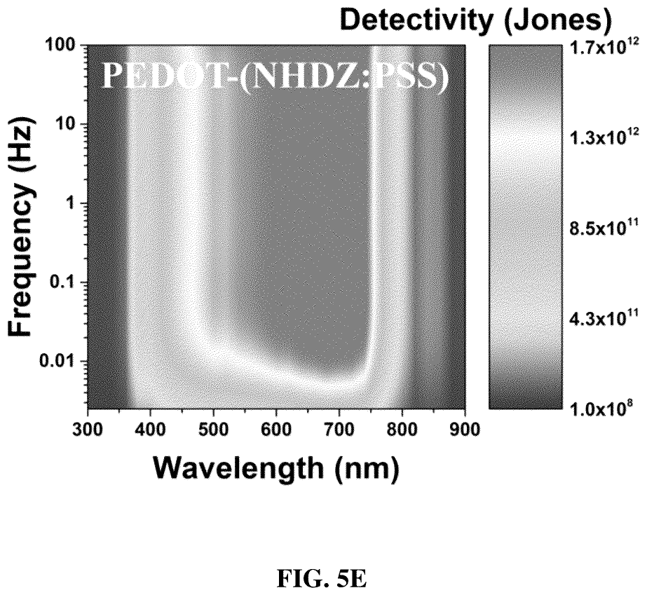

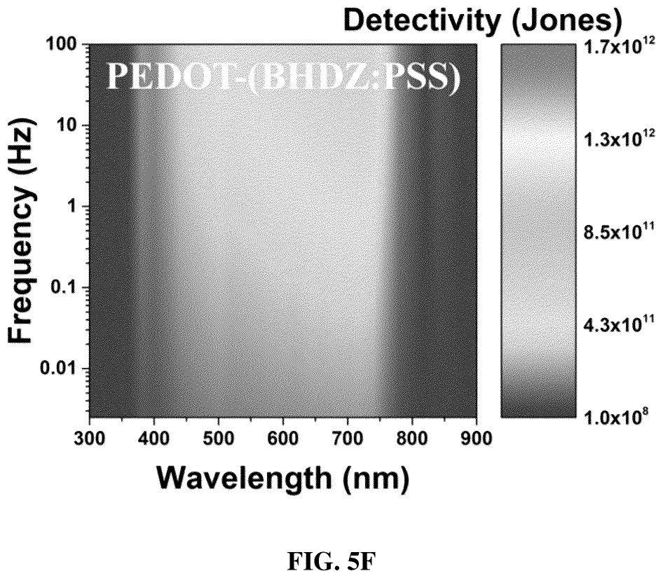

FIGS. 5A-5F are a graph showing the noise spectrum of the instrument background and OPD (FIG. 5A), a graph showing the comparison of thermal and shot noise for the OPD (FIG. 5B), a graph showing the noise equivalent power (NEP) obtained at 1 Hz (FIG. 5C), and a 2D contour plot showing the specific detection rate (D*) as a function of the incident wavelength and frequency (FIGS. 5D-5F).

Table 5 shows the noise components, noise spectrum, NEP, specific detectability, and shot noise limited specific detectability parameters of the OPD.

TABLE 5

| | Sₙ @ 1 Hz | Shot Noise | Thermal noise | NEP @1 Hz | D* @ 1 Hz | Dₛₕ* |
|---|---|---|---|---|---|---|
| Each OPD | (A Hz^{-1/2}) | (A Hz^{-1/2}) | (A Hz^{-1/2}) | (W Hz^{-1/2}) | (Jones) | (Jones) |
| PEDOT-(PSS) | 2.33 × 10⁻¹³ | 7.84 × 10⁻¹⁵ | 2.57 × 10⁻¹⁴ | 4.55 × 10⁻¹³ | 8.01 × 10¹¹ | 1.96 × 10¹³ |
| PEDOT-(NHDZ:PSS) | 9.04 × 10⁻¹⁴ | 6.25 × 10⁻¹⁵ | 2.11 × 10⁻¹⁴ | 1.99 × 10⁻¹³ | 1.95 × 10¹² | 2.58 × 10¹³ |
| PEDOT-(BHDZ:PSS) | 3.38 × 10⁻¹³ | 7.59 × 10⁻¹⁵ | 4.23 × 10⁻¹⁴ | 8.76 × 10⁻¹³ | 4.41 × 10¹¹ | 1.97 × 10¹³ |

In FIG. 5A, the noise density spectrum of the OPD was directly measured without applying a bias voltage.

FIG. 5B shows that the thermal noise of the OPD is ≈10⁻¹⁴ A HZ^{-0.5}, which is about 10 times higher than that of the shot noise (≈10⁻¹⁵ A HZ^{-0.5}). Thermal noise shows the inaccuracy of calculating the detection rate with shot noise alone by replacing shot noise with the dominant noise source.

The noise spectral density at 1 Hz is greater than or equal to 9.04×10⁻¹⁴ A Hz^{-1/2}, specifically 9.04×10⁻¹⁴ to 3.38×10⁻¹ A Hz^{-1/2}, and the lowest value of 9.04×10⁻¹⁴ A Hz^{-1/2} when optimized is 258% lower than that of the conventional one.

The shot noise may be greater than or equal to 6.25×10⁻¹⁵ A Hz^{-1/2}, and specifically 6.25×10⁻¹⁵ A Hz^{-1/2} to 7.84×10⁻¹⁵ A Hz^{-1/2}, and the minimum value being the optimum value is a value further reduced by 125% compared to that of the conventional one.

The thermal noise may be greater than or equal to 2.11×10⁻¹⁴ A Hz^{-1/2}, and specifically 2.11×10⁻¹⁴ A Hz^{-1/2} to 4.23×10⁻¹⁴ A Hz^{-1/2}, and the minimum value being the optimum value is a value further reduced by 122% compared to that of the conventional one.

The NEP values of each OPD at a frequency of 1 Hz correspond to 4.55×10⁻¹³ WHz^{-1/2} (PEDOT-(PSS)), 1.99×10⁻¹³ WHz^{-1/2} (PEDOT-(NHDZ:PSS)), and 8.76×10⁻¹³ WHz^{-1/2} (PEDOT-(BHDZ:PSS)), respectively. The curve is shown in FIG. 5C. The increase in NEP in other devices compared to the optimal device may be explained as an increase in 1/f noise as the operating frequency decreases.

FIGS. 5D, 5E and 5F show a 2D contour plot of the specific detection rate (D*), where the specific detection rate (cm Hz^{1/2} W⁻¹) is the reciprocal of NEP normalized by the square root of the active area and may be calculated by the equation D*=\sqrt{A}/NEP=R\sqrt{A}/S_{W}*. For each OPD, the corresponding D* at 1 Hz is 8.01×10¹¹ cm Hz^{1/2} W⁻¹ (PEDOT-(PSS)) and 4.41×10¹¹ cm Hz^{1/2} W⁻¹ (PEDOT-(BHDZ:PSS)). Optimized OPD based on PEDOT-(NHDZ:PSS) shows an improvement of 243%/442% higher in D*(1.95×10¹² cm Hz^{1/2} W⁻¹). The improved performance may be mainly due to an increase in shunt resistance and a corresponding decrease in volume for heat generation by the carriers.

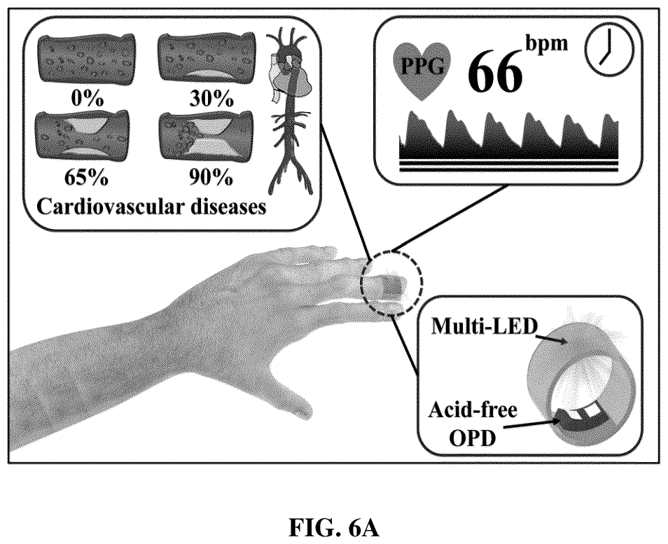

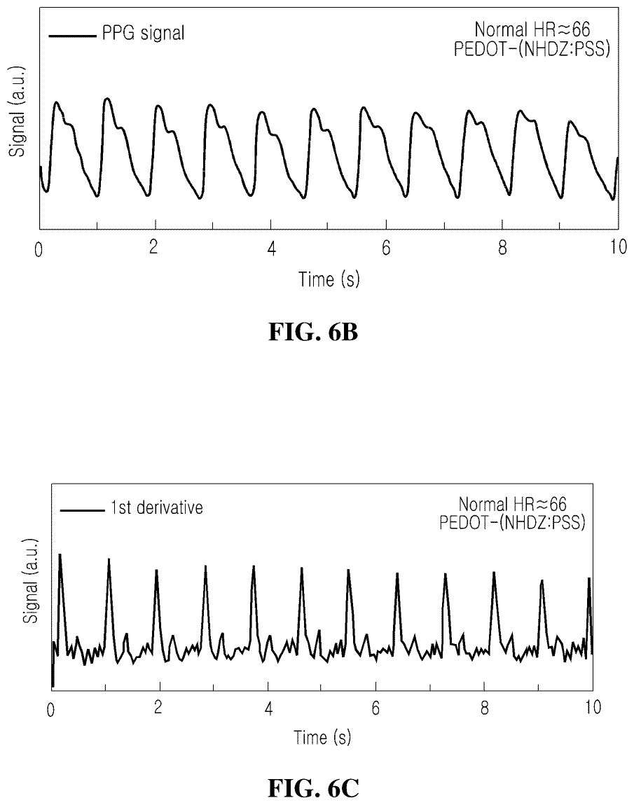

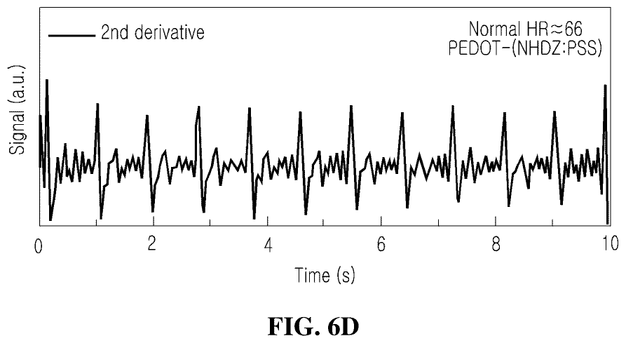

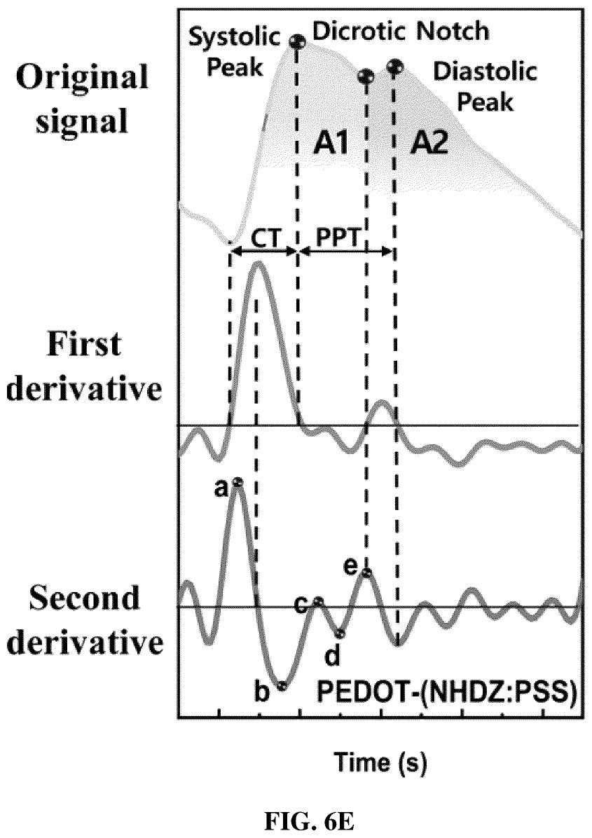

FIGS. 6A-6E are a schematic view of the acid-free OPD-based PPG sensor for diagnosing cardiovascular diseases (FIG. 6A), graphs showing the comparison of the original (FIG. 6B), first derivative (FIG. 6C), and second derivative (FIG. 6D) of PPG signals measured using PEDOT-(NHDZ:PSS)-based OPD, and an enlarged curve showing FIGS. 6B-6D for extracting features in the cardiac cycle (FIG. 6E).

Referring to FIG. 6A, a PPG sensor for blood circulation measurement and diagnosis of the cardiovascular diseases using a multi-wavelength LED as a light source may be confirmed.

The first derivative of the PPG waveform was performed to obtain the VPG curve and is shown in FIG. 6C. FIG. 6E shows an enlarged version of FIGS. 6B, 6C and 6D for easier observation. The starting point, systolic peak, hyperbolic notch, and diastolic peak of the original PPG waveform are defined as the four points closest to the baseline in the VPG. The ratio of the two regions (systolic peak-containing A1 and diastolic peak-containing A2) segmented from the hyperbolic notch is known as the inflection and harmonic area ratio (IHAR), which is used to continuously monitor the cardiac output (CO). The time from the beginning of the original PPG waveform to the systolic peak is called the crest time (CT) and is a useful feature for cardiovascular disease classification. The time from the systolic peak to the diastolic peak of the PPG waveform is called the peak-to-peak time (PPT). This is related to the time it takes for blood released from the heart to pass through the surrounding vasculature. At the same time, PPT is used to determine whether blood circulation is healthy. Four independent cardiac cycles were considered herein. This was obtained from a 24-year-old healthy male subject. It is widely regarded as the most effective feature of PPG primary derivation for accurate cardiovascular disease classification. These features were successfully measured using an optimized OPD.

The foregoing description of the present invention is intended to be illustrative, and it will be understood by those skilled in the art that other specific forms may be easily modified without changing the technical idea or essential features of the present invention. It should therefore be understood that the embodiments described above are illustrative in all respects and not restrictive. For example, each component described as being unitary may be implemented in a distributed manner, and similarly, components described as being distributed may also be implemented in a combined manner.

The scope of the present invention is indicated by the claims described below, and all changes or modifications derived from the meaning and scope of the claims and their equivalent concepts should be construed as being included in the scope of the invention.

What is claimed is:

1. An organic photodiode comprising an acid-free hole transport layer, having minimized acidity and improved nanostructure of a film, thereby reducing trap density and improving charge carrier mobility.

2. The organic photodiode comprising the acid-free hole transport layer of claim 1, wherein the acid-free hole transport layer includes a PEDOT-(PSS) film to which a heterocyclic 1,3-diazole is bonded.

3. The organic photodiode comprising the acid-free hole transport layer of claim 2, wherein the heterocyclic 1,3-diazole forms a hydrogen bond with the PEDOT-(PSS) film to reduce a Coulomb force in the PEDOT-(PSS) film and reorient a core-shell structure into a linear chain, thereby improving hole transport capability.

4. The organic photodiode comprising the acid-free hole transport layer of claim 1, wherein the organic photodiode has a root-mean-squared (RMS) value of roughness of at least 0.381 nm.

5. The organic photodiode comprising the acid-free hole transport layer of claim 1, wherein the organic photodiode has an optical bandgap of 1.97 to 2.04 eV.

6. The organic photodiode comprising the acid-free hole transport layer of claim 1, wherein the organic photodiode has a carrier mobility of 1.95×10⁻³ to 5.02×10⁻³ cm² V⁻¹ s⁻¹.

7. The organic photodiode comprising the acid-free hole transport layer of claim 1, wherein the organic photodiode has a polymerization degree of 78.79 to 91.04%.

8. The organic photodiode comprising the acid-free hole transport layer of claim 1, wherein the organic photodiode has a LUMO of −3.08 to −3.13 eV.

9. The organic photodiode comprising the acid-free hole transport layer of claim 1, wherein the organic photodiode has a dark current of 3.91×10⁻⁹ A cm⁻² to 4.11×10⁻⁸ A cm⁻².

10. A method for manufacturing an organic photodiode, comprising:
preparing a mixed solution of a PEDOT-(PSS) solution and a heterocyclic 1,3-diazole solution;
coating the mixed solution onto a substrate to form a mixed solution layer;
coating an active layer solution onto the mixed solution layer to form an active layer; and
depositing an electrode onto the active layer.

11. The method for manufacturing the organic photodiode of claim 10, wherein the active layer solution is PBDTTT-EFT:PC₇₀BM.

12. A photodetection sensor comprising the organic photodiode of claim 1.

13. The photodetection sensor of claim 12, wherein the photodetection sensor has a photodetection capacity (D*@1 Hz) of 4.41×10¹¹ to 1.95×10¹² Jones.

14. The photodetection sensor of claim 12, wherein the photodetection sensor has an optical signal bandwidth (f_{3dB}) of 192.54 to 260 kHz.

15. The photodetection sensor of claim 12, wherein the photodetection sensor has a response time (light on) of 0.96 to 4.45 μs.

16. A method of photoplethysmography using the photodetection sensor of claim 12.

17. The method of photoplethysmography of claim 16, wherein performance of photoplethysmography is improved due to selectivity for pink LEDs in multiple wavelength bands.

18. The method of photoplethysmography of claim 16, wherein detection is possible based on composite noise of noise spectral density, shot noise, and thermal noise at 1 Hz.

19. The method of photoplethysmography of claim 18, wherein the noise spectral density at 1 Hz is 9.04×10⁻¹⁴ to 3.38×10⁻¹³ A Hz^{-1/2}.

20. The method of photoplethysmography of claim 18, wherein the shot noise at 1 Hz is 6.25×10⁻¹⁵ A Hz^{-1/2} to 7.84×10−¹⁵ A Hz^{-1/2}.

21. The method of photoplethysmography of claim 18, wherein the thermal noise at 1 Hz is 2.11×10⁻¹⁴ A Hz^{-1/2} to 4.23×10⁻¹⁴ A Hz^{-1/2}.

22. The method of photoplethysmography of claim 16, comprising extracting features by specific locations in a waveform through an acceleration plethysmogram and calculating them to diagnose cardiovascular disease conditions and determine blood circulation.
